(19) United States
(12) Patent Application Publication
(10) Pub. No.: US 2026/0011403 A1
(43) Pub. Date: Jan. 8, 2026

(54) DETECTING AND GENOTYPING VARIABLE NUMBER TANDEM REPEATS

(71) Applicant: Illumina, Inc., San Diego, CA (US)

(72) Inventors: Marzieh Eslami Rasekh, Carlsbad, CA (US); Jeffrey Yuan, Long Island City, NY (US); Sean Truong, San Diego, CA (US)

(21) Appl. No.: 18/869,134

(22) PCT Filed: Sep. 19, 2023
(86) PCT No.: PCT/US2023/074604

§ 371 Date: Nov. 25, 2024

Related U.S. Application Data
(60) Provisional application No. 63/377,172, filed on Sep. 26, 2022.

(51) Int. Cl. G16B 20/10; G16B 30/10

(52) U.S. Cl. CPC G16B 20/10; G16B 30/10

(57) ABSTRACT

Disclosed herein are methods and systems for determining a score for the copy number of repeat units in a variable number tandem repeat (VNTR) locus in a target polynucleotide. Also disclosed herein are methods and systems for determining the nucleotide sequence of a sample nucleic acid having repeat units, where the methods and systems may utilize the most likely copy number of repeat units determined according to the aforementioned methods and systems. Also disclosed herein are methods and systems for predicting a feature of a subject, wherein the methods and systems may utilize the score for the copy number of repeat units in a VNTR locus in a target polynucleotide determined according to the aforementioned methods and systems.

### CROSS-REFERENCE TO RELATED APPLICATIONS

This application is a U.S. National Phase Application of PCT International Application Number PCT/US2023/074604, filed Sep. 19, 2023, which claims priority to U.S. Provisional Patent Application No. 63/377,172, filed Sep. 26, 2022 entitled DETECTING AND GENOTYPING VARIABLE NUMBER TANDEM REPEATS, the disclosure of which is incorporated herein by reference in its entirety.

### REFERENCE TO SEQUENCE LISTING

The present application is being filed along with a Sequence Listing in electronic format. The Sequence Listing is provided as a file entitled ILLINC731WO, created Sep. 19, 2023, which is approximately 2030 bytes in size. The information in the electronic format of the Sequence Listing is incorporated herein by reference in its entirety.

### BACKGROUND

### Field

The disclosed technology relates to the field of nucleic acid sequencing. More particularly, the disclosed technology relates to detecting and genotyping variable number tandem repeats in a sample nucleic acid utilizing paired-end nucleic acid sequencing techniques.

### Description of the Related Art

Variable number tandem repeats (VNTRs) account for a significant proportion of between-genome variation. Accurate detection of VNTRs has long been complicated by the low-complexity nature of VNTR regions and the large size of the repetitive sequences in VNTRs. There exists a continuing need for improving the detection and characterization of VNTRs in nucleic acid sequencing technologies.

### SUMMARY

In some aspects, disclosed herein are systems and methods for accurately characterizing the variations present in Tandem Repeat (TR) regions in a genome (e.g., the human genome). Disclosed systems and methods may involve the use of non-parametric hypothesis testing, likelihood modeling, and/or genome assembly. In some embodiments, the disclosed systems and methods may be applicable to VNTRs smaller than the average fragment size of a sequencing read. In some embodiments, the disclosed systems and methods may utilize the distributions of the fragment sizes to determine the copy number of repeat units in a VNTR. In some embodiments, the disclosed systems and methods may further utilize the presence of single nucleotide variants (SNVs) and/or indels in a VNTR to reconstruct the full target/unknown VNTR array sequence. In some embodiments, the disclosed systems and methods may characterize both variations in the repeat unit copy number (e.g., repeat unit deletions and duplications) and small variants present in the VNTR (SNVs and/or indels).

Although the examples herein concern humans and the language is primarily directed to human concerns, the concepts described herein are applicable to genomes from any biological entities, such as plants or animals. The nucleic acid samples can be derived from cells, cell-free DNA, cell-free fetal DNA, amniotic fluid, a blood sample, a biopsy sample, or a combination thereof. The sequence reads can be generated by short-read, paired-end sequencing technologies. The sequence reads can be generated by targeted sequencing or whole genome sequencing (WGS). In some embodiments, the WGS can be clinical WGS (cWGS).

In one aspect, the disclosed technology relates to a method for determining a score for the copy number of repeat units in a variable number tandem repeat (VNTR) locus in a target polynucleotide, comprising: obtaining a plurality of paired-end sequence reads, wherein each paired-end sequence read is associated with a nucleic acid fragment that spans the VNTR locus in the target polynucleotide; obtaining the alignment, against a reference sequence, of each paired-end sequence read associated with a spanning region of the VNTR locus; determining the length of the nucleic acid fragment associated with each paired-end sequence read; calculating a first distribution of the lengths of the nucleic acid fragments; determining secondary distributions of the lengths of mapped segments in normalization regions for a plurality of copy numbers associated with the plurality of paired-end sequence reads; and comparing the first distribution with at least one of the secondary distributions to generate a score of the copy number of repeat units in the VNTR locus in the target polynucleotide.

In some embodiments, the normalization regions are evolutionarily conserved regions in the reference sequence and/or are regions in the reference sequence that do not comprise structural variation events. In some embodiments, obtaining the plurality of paired-end sequence reads comprises selecting, from a set of sequence reads, a subset of sequence reads that align to the VNTR locus with an alignment score higher than a threshold. In some embodiments, the alignment tolerates a degree of mismatch lower than a threshold. In some embodiments, the method further comprises confirming that at least 40 paired-end sequence reads align to the VNTR locus in the reference sequence. In some embodiments, the method further comprises confirming that at least 50 paired-end sequence reads align to the VNTR locus in the reference sequence.

In some embodiments, the method further comprises determining a statistical significance associated with the copy number of repeat units in the VNTR locus in the target polynucleotide, based in part on the number of paired-end sequence reads that align to the VNTR locus in the reference sequence. In some embodiments, the method further comprises determining a statistical significance (e.g., p-value) associated with the copy number of repeat units in the VNTR locus in the target polynucleotide, based in part on how well the first distribution fits to the secondary distributions. In some embodiments, the first distribution is compared with the at least one of the secondary distributions by way of a non-parametric probability calculation. In some embodiments, the secondary distributions are determined by statistical modeling and compared to the first distribution by statistical tests. In some embodiments, the secondary distributions are determined based in part on the plurality of copy numbers, the pattern of the VNTR locus, and copy number of the VNTR locus. In some embodiments, comparing the first distribution with the at least one of the secondary distributions comprises comparing a posterior probability for each genotype. In some embodiments, a repeat unit is longer than about 10 base pairs in length.

In some embodiments, the VNTR locus is about 300 base pairs to about 600 base pairs in length. In some embodiments, the VNTR locus is part of a macrosatellite or a minisatellite. In some embodiments, the macrosatellite has repeat patterns of longer than 100 base pairs in length. In some embodiments, the minisatellite has repeat patterns of about 10 base pairs to about 100 base pairs in length. In some embodiments, each paired-end sequence read is about 100 base pairs to about 500 base pairs in length. In some embodiments, the plurality of paired-end sequence reads is generated by targeted sequencing, whole genome sequencing (WGS), or clinical WGS. In some embodiments, the plurality of paired-end sequence reads is generated by a next generation sequencing reaction. In some embodiments, the target polynucleotide is extracted from cells, a cell-free DNA sample, an amniotic fluid, a blood sample, a biopsy sample, or a combination thereof. In some embodiments, the target polynucleotide is from a human subject and the reference sequence is a portion of a consensus human genome or transcriptome. In some embodiments, the method further comprises performing paired-end sequencing of a plurality of copies of the target polynucleotide to obtain the plurality of paired-end sequence reads, wherein each paired-end sequence read is obtained from a nucleic acid cluster on a solid substrate. In some embodiments, the method further comprises performing bridge amplification of the target polynucleotide to provide copies of the target polynucleotide in a nucleic acid cluster on a solid substrate.

In another aspect, the disclosed technology relates to a system for determining a score for the copy number of repeat units in a variable number tandem repeat (VNTR) locus in a target polynucleotide, comprising: a nucleic acid sequencer; a non-transitory memory configured to store executable instructions; and a hardware processor in communication with the nucleic acid sequencer and the non-transitory memory, the hardware processor programmed by the executable instructions to perform the methods disclosed herein for determining a score for the copy number of repeat units in a variable number tandem repeat (VNTR) locus in a target polynucleotide. In some embodiments, the non-transitory memory is configured to store the reference sequence. In some embodiments, the hardware processor is configured to obtain the reference sequence from an external database. In some embodiments, the hardware processor is configured to receive the plurality of paired-end sequence reads from the nucleic acid sequencer. In some embodiments, the hardware processor is configured to control the nucleic acid sequencer to perform sequencing of the target polynucleotide. In some embodiments, the hardware processor is configured to control the nucleic acid sequencer to perform additional sequencing of the target polynucleotide based on the determined score for the copy number of repeat units in the VNTR locus in the target polynucleotide. In some embodiments, the hardware processor is configured to output, on a display, the most likely copy number of repeat units in the VNTR locus in the target polynucleotide and/or an associated score.

In another aspect, the disclosed technology relates to a method for predicting a feature of a subject, comprising: forming a data element including a score for the copy number of repeat units in a variable number tandem repeat (VNTR) locus in a target polynucleotide from a subject determined by the methods disclosed herein for determining a score for the copy number of repeat units in a variable number tandem repeat (VNTR) locus in a target polynucleotide; and applying a trained machine learning or statistical model to the data element to predict a feature of the subject. In some embodiments, the method further comprises: including in the data element, the most likely copy number of repeat units in the VNTR locus in the target polynucleotide, a distance between the VNTR locus in the target polynucleotide and a gene in the genome of the subject, the length of a repeat unit, the GC content of the VNTR locus in the target polynucleotide, the degree of mismatch in the alignment, and/or the probability that the VNTR locus in the target polynucleotide has mutated. In some embodiments, the feature is an identity or a disease of the subject.

In another aspect, the disclosed technology relates to a method for determining the nucleotide sequence of a sample nucleic acid having repeat units, comprising: obtaining a first plurality of paired-end sequence reads that each aligns and spans a variable number tandem repeat (VNTR) locus in a reference sequence, and a copy number of repeat units in the sample nucleic acid; determining the consensus pattern motif and the positions of the first plurality of paired-end sequence reads with respect to the VNTR locus, the consensus pattern motif comprising a plurality of events of single nucleotide variants or indels; determining, based on the consensus pattern motif and the positions of the first plurality of paired-end sequence reads with respect to the VNTR locus, which repeat unit of the copy number of repeat units an event occurs in; and constructing the nucleotide sequence based in part on the copy number of repeat units, and which repeat unit of the copy number of repeat units the event occurs within.

In some embodiments, the copy number of repeat units in the sample nucleic acid is the most likely copy number of repeat units determined base in part on the methods disclosed herein for determining a score for the copy number of repeat units in a variable number tandem repeat (VNTR) locus in a target polynucleotide. In some embodiments, determining the consensus pattern motif and the positions of the first plurality of paired-end sequence reads with respect to the VNTR locus comprises remapping the first plurality of paired-end sequence reads to the VNTR locus by a circular alignment process. In some embodiments, the method further comprises: obtaining the alignments for the first plurality of paired-end sequence reads; and determining, prior to the remapping, whether single nucleotide variants or indels occur in the first plurality of paired-end sequence reads based on the obtained alignments.

In some embodiments, determining which repeat unit of the copy number of repeat units the event occurs in comprises: identifying constituent sequence reads of the first plurality of paired-end sequence reads that overlap the event; calculating: an observed left distribution of the start positions of the mates of the constituent sequence reads that map to a left flanking region of the VNTR locus, and an observed right distribution of the start positions of the mates of the constituent sequence reads that map to a right flanking region of the VNTR locus; generating, assuming that the event occurs in the j′th repeat unit of the copy number of repeat units, an expected left distribution of the start positions of the mates of the constituent sequence reads that map to a left flanking region of the VNTR locus, and an expected right distribution of the start positions of the mates of the constituent sequence reads that map to a right flanking region of the VNTR locus; evaluating: the similarity between the observed left distribution and the expected left distribution, and the similarity between the observed right distribution and the expected right distribution; and determining, based on the evaluation, the likelihood of the event occurring in the j′th repeat unit of the copy number of repeat units.

In some embodiments, evaluating the similarity between the distributions is by way of a statistical test. In some embodiments, evaluating the similarity between the distributions is by way of the posterior probability of each possible genotype. In some embodiments, the expected left distribution and the expected right distribution are generated by statistical modeling. In some embodiments, the expected left distribution is a distribution of the lengths of normalization sequences in the reference sequence that are greater than the distance from the event to the left flank region of the VNTR, given that the event occurs in the j′th repeat unit. In some embodiments, the normalization sequences are evolutionarily conserved regions in the reference sequence or regions in the reference sequence that do not comprise structural variation events. _ In some embodiments, the expected right distribution is a distribution of the lengths of normalization sequences in the reference sequence that are greater than the distance from the event to the right flank region of the VNTR, given that the event occurs in the j′th repeat unit. In some embodiments, the normalization sequences are evolutionarily conserved regions in the reference sequence or regions in the reference sequence that do not comprise structural variation events.

The systems, devices, kits, and methods disclosed herein each have several aspects, no single one of which is solely responsible for their desirable attributes. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

It is to be understood that any features of the systems disclosed herein may be combined in any desirable manner and/or configuration. Further, it is to be understood that any features of the methods disclosed herein may be combined in any desirable manner. Moreover, it is to be understood that any combination of features of the methods and/or the systems may be used together, and/or may be combined with any of the examples disclosed herein. It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A, FIG. 1B and FIG. 1C show non-limiting exemplary illustrations of a VNTR in a reference sequence.

FIG. 2 schematically illustrates an example spanning read pairs with respect to a VNTR in a reference sequence.

FIG. 3A and FIG. 3B are flow diagrams that schematically illustrate methods of detecting and genotyping VNTRs according to some embodiments of the disclosed technology.

FIGS. 4A and FIG. 4B schematically illustrate that an exemplary distribution of the spanning fragment sizes varies with single copy insertions and deletions in VNTRs.

FIG. 4C and FIG. 4D illustrate exemplary results of extraction of spanning fragments for a target VNTR region.

FIG. 4E is a bar graph illustrating the modeling of a spanning fragment size distribution from the results from FIGS. 4C and 4D.

FIG. 4F, FIG. 4G and FIG. 4H are bar charts which illustrate exemplary results of calculating secondary distributions for several possible copy numbers based on a set of normalization regions.

FIG. 5 illustrates the circular alignment of two reads to a consensus VNTR pattern according to some examples of the disclosed technology.

FIG. 6A is a block diagram of an exemplary sequencing system that may be used to perform the disclosed methods.

FIG. 6B is a block diagram of an exemplary computing device that may be used in connection with the exemplary sequencing system of FIG. 6A.

FIGS. 7A, 7B and 7C illustrate an example process of resolving the haplotype sequence.

### DETAILED DESCRIPTION

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

### Variable Number Tandem Repeats

Variable number tandem repeats (VNTRs) are a class of structural variants that include tandem repeats of patterns, for example patterns larger than 10 base pairs (bps), and that differ in copy number among the genomes of individuals of a species. While VNTRs cover <5% of the human genome, about 50% of all structural variants (variants greater than 50 bp) are VNTRs. In some cases, a VNTR can have fewer than 20% mismatches for an exact repeat. In some cases, VNTRs can have small variants, such as SNPs and indels in the repetitive sequences. On average one person has about 2.2 mega base pairs (Mbps) of deleted sequence and about 5.7 Mbps of inserted sequence in VNTRs. Variations in VNTRs can depend on the populations within a species.

Some VNTRs are known to be associated with genetic diseases, such as bipolar disorder, MCKD1, stroke, CAD, FSHD, ADHD, Parkinson's, diffuse panbronchiolitis (DPB), monogenic diabetes, TID, T2D, obesity, OCD, osteochondritis dissecans, Kawasaki, ATF in stroke, BPSD, Alzheimer's, anxiety, schizophrenia, metastatic colorectal cancer, Kawasaki, or progressive myoclonic epilepsy 1A. A VNTR can be present in the coding region or non-coding region. Moreover, a VNTR can be present in the 5′ untranslated region (UTR), promoter, intron, or 3′ UTR. The gene that includes, or is affected by, the VNTR can be, for example, PER3, MUC1, IL1RN, DUX4, DAT1, MUC21, CEL, INS, DRD4, ACAN, ZFHX3, GP1BA, SERT, SERT, HIC1, MMP9, CSTB, or MAOA.

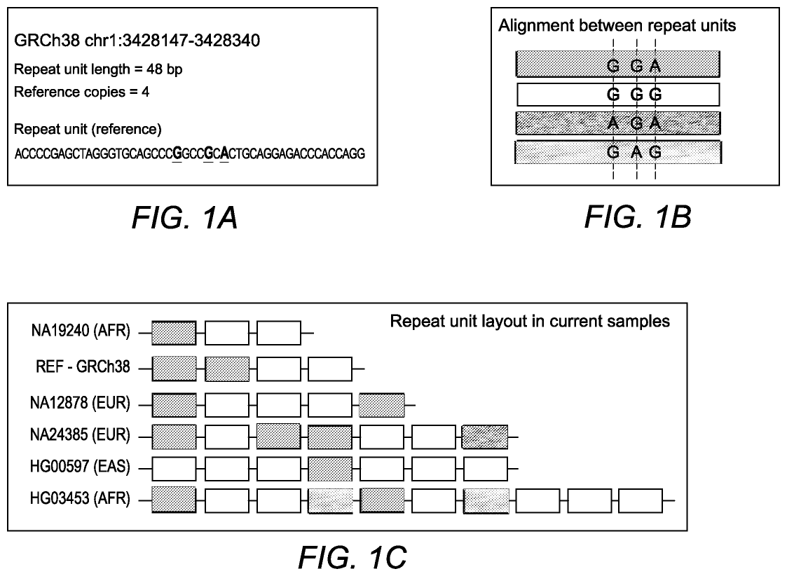

FIG. 1A, FIG. 1B and FIG. 1C show a non-limiting exemplary illustration of a VNTR in a reference sequence. FIG. 1A shows that a VNTR in the reference human genome GRCh38 is at chr1: 3428147-3428340. The repeat unit has a length of 48 bps. The reference sequence of repeat the unit is ACCCCGAGCTAGGGTGCAGCCCGGCCGCACTGCAGGAGACCCACCAGG (SEQ ID NO: 1) in GRCh38. Different copies of the repeat unit in the VNTR (within a haplotype or across haplotypes) can vary, in particular at the three bases bolded and underlined. FIG. 1B shows that the three bases can be G, G, and A, respectively, in a first type or sequence of the repeat unit; G, G, and G, respectively, in a second type or sequence of the repeat unit; A, G, and A, respectively, in a third type or sequence of the repeat unit; and G, A, and G, respectively, in a fourth type or sequence of the repeat unit. FIG. 1C show that the VNTR includes four copies of the repeat unit in GRCh38. The four copies include two copies of the first type followed by two copies of the second type. The five samples included three, five, seven, seven, and ten copies of the repeat unit, respectively. For sample NA19240 of a subject who is African, the VNTR included one copy of the first type followed by two copies of the second type. For sample NA12878 of a subject who is European, the VNTR included one copy of the first type, three copies of the second type, and one copy of the first type. For sample NA24385 of a subject who is European, the VNTR included one copy of the first type, one copy of the second type, two copies of the first type, two copies of the second type, and one copy of the third type. For sample HG00597 of a subject who is Eastern Asian, the VNTR included three copies of the second type, one copy of the first type, and three copies of the second type. For sample HG03453 of a subject who is African, the VNTR included one copy of the first type, two copies of the second type, one copy of the fourth type, one copy of the first type, one copy of the second type, one copy of the fourth type, and three copies of the second type. The examples discussed in connection with FIGS. 1A, 1B and 1C pertain to homozygous variants where both alleles include the VNTR locus.

The difficulty of detecting VNTRs is multi-dimensional. The nature of the tandem repeats causes low mappability and high sequencing errors. Existing sequencing techniques (including, for example, using population haplotypes in the genome graph) suffer from low precision in detecting VNTRs due to the repetitive nature of VNTRs. Short-read sequencing technologies have a higher throughput compared to long-read sequencing technologies, but short sequencing reads often cannot cover the full length of most VNTRs. For example, around 29% of the VNTRs have additional repeats with total length greater than or equal to 150 bps in one individual. Due to the repetitive nature of VNTRs, correctly rebuilding VNTRs' haplotypes from short reads is difficult. With short sequencing reads, methods of detecting VNTRs may utilize the read sequences and some form of circular alignment (or wrap-around alignment) to infer the copy number changes in tandem repeats; however, these methods only allow for genotyping of small VNTRs (i.e., smaller than the read length). Abnormal fragment sizes—a read pair that maps beyond the normal distribution—have been used in the prior art to infer some classes of large structural variants such as large changes in VNTRs; however, some VNTRs may not be accurately detected by this approach if the VNTRs are shorter compared to the variance in the insert size of the sequencing reads. For example, VNTRs may not be accurately detected with paired-end sequencing reads, which have a high variance in insert size. Moreover, using existing methods, local reassembly of the VNTR sequences is difficult and often fails. Therefore, there is a need for improved methods for detecting and genotyping VNTRs.

### Overview

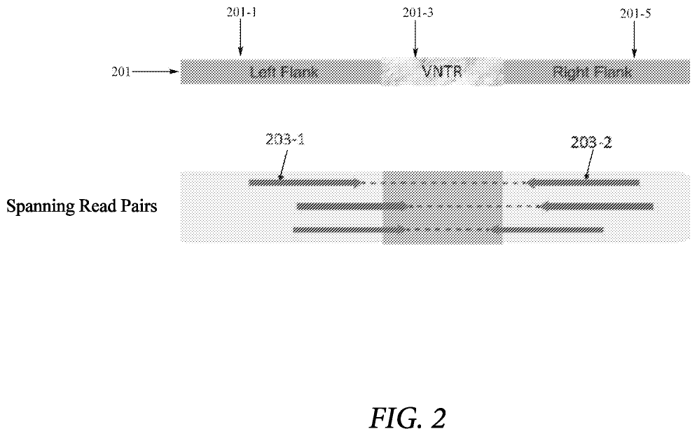

The disclosed systems and methods of detecting and genotyping VNTRs overcome the problem of large insert size variance by examining read pairs (from paired-end sequencing) that “span” a VNTR region and comparing the number of fragments and their sizes against the expected sizes. FIG. 2 schematically illustrates one example of spanning read pairs with respect to a VNTR 201-3 in a reference sequence 201. The reference sequence 201 also includes the left flank region 201-1 and the right flank region 201-5 that are on the two ends of the VNTR 201-3. An example spanning read pair includes the left read 203-1 and the right read 203-2. A spanning read pair originates from a nucleic acid fragment that completely spans the VNTR, such that each read in the read pair maps on either end of the VNTR, i.e., the left read and the right read are on the left flank and the right flank of the VNTR, respectively. “Left” and “right” in the context of the disclosed technology are defined with respect to the direction that letters representing a polynucleotide sequence is read.

The read pairs may be obtained from the paired-end sequencing process as described in U.S. Pat. No. 10,329,613, the disclosure of which is incorporated herein by reference, or other paired-end sequencing technologies. The disclosed systems and methods may use a maximum likelihood model in addition to a Bayesian model to predict the most likely genotype of the VNTR. The disclosed systems and methods may determine the number of repeats in a VNTR and/or reconstruct the full sequence of each target/unknown VNTR array.

In some embodiments, the disclosed methods of detecting and genotyping VNTRs may include two processes. In the first process, the copy number of each VNTR may be determined by comparing the observed fragment size distribution of a sample to the corresponding expected fragment size distribution from background/normalization regions with no expected SV events. In particular, the comparison may be performed by generating the expected fragment size distributions for 0 or 1 or more SV events from the background/normalization regions, and comparing the generated expected fragment size distributions with the observed distribution. A non-parametric test may be used to choose the most likely copy number based on the observed and expected distributions. In the second process, detected SNVs and/or indels in the VNTR may be used to reconstruct the full sequence for the repeat units in the VNTR by determining the phasing and location of the SNVs and/or indels in the target/unknown VNTR array. The output of the disclosed method may be the copy number of the VNTR and if applicable, the full, haplotype-resolved SNV and indel locations in the target/unknown VNTR array. In some embodiments, the second process may utilize the results of the first process.

Since the disclosed systems and methods examine spanning read pairs instead of reads containing the VNTR, the disclosed systems and methods were found to result in highly accurate VNTR genotyping (e.g., determining the number of repeats in a VNTR with a high accuracy and/or reconstructing the full sequence of each target/unknown VNTR array with a high accuracy). The spanning reads align at least partially outside of the VNTR repeat region and therefore are not negatively impacted by the VNTR repeat sequence, which suffers from high sequencing and mapping errors. The disclosed systems and methods can detect large VNTRs, for example, VNTRs in length up to the insert size (e.g., 350 bp, 400 bp, 450 bp, 500 bp, 550 bp, 600 bp, 650 bp, 700 bp, 750 bp, 800 bp, 850 bp, 900 bp, 950 bp, 1000 bp, 2000 bp, 3000 bp, 4000 bp, 5000 bp or longer). The disclosed systems and methods can improve the recall (also known as sensitivity, the percentage of true variants that are correctly detected) of structural variants by 20%, 50%, 80%, 100% or more. The disclosed systems and methods can detect VNTRs up to the length of the paired-end fragment size using short-read sequencing technologies, with a performance (e.g., recall and/or precision) on VNTRs shorter 0.7 fragment size that is comparable to the performance when long-read sequencing technologies are used. Since short-read sequencing technologies have a higher throughput compared to long-read sequencing technologies, the disclosed systems and methods can detect and characterize VNTRs more efficiently. As many VNTRs play functional roles in the human cells and regulation of genes, the disclosed systems and methods have clinical applications in disease diagnosis and treatment.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides may be natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single-or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

The term “primer,” as used herein refers to an isolated oligonucleotide that is capable of acting as a point of initiation of synthesis when placed under conditions inductive to synthesis of an extension product (e.g., the conditions include nucleotides, an inducing agent such as DNA polymerase, and a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, use of the method, and the parameters used for primer design.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

A “genome” refers to the complete genetic information of an organism or virus, expressed in nucleic acid sequences.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nlm.nih.gov. In various embodiments, the reference sequence is significantly larger than the reads that are aligned to it. For example, it may be at least about 100 times larger, or at least about 1000 times larger, or at least about 10,000 times larger, or at least about 105 times larger, or at least about 106 times larger, or at least about 107 times larger. In one example, the reference sequence is that of a full-length genome. Such sequences may be referred to as genomic reference sequences. For example, the reference sequence can be a reference human genome sequence, such as hg19 or hg38. In another example, the reference sequence is limited to a specific human chromosome such as chromosome 13. In some embodiments, a reference Y chromosome is the Y chromosome sequence from human genome version hg19. Such sequences may be referred to as chromosome reference sequences. Other examples of reference sequences include genomes of other species, as well as chromosomes, sub-chromosomal regions (such as strands), etc., of any species. In various embodiments, the reference sequence is a consensus sequence or other combination derived from multiple individuals. However, in certain applications, the reference sequence may be taken from a particular individual.

The term “nucleic acid sample” herein refers to a sample, typically derived from a biological fluid, cell, tissue, organ, or organism, comprising a nucleic acid or a mixture of nucleic acids comprising at least one nucleic acid sequence that is to be screened for copy number variation. In certain embodiments the nucleic acid sample comprises at least one nucleic acid sequence whose copy number is suspected of having undergone variation. Such samples may include, but are not limited to sputum/oral fluid, amniotic fluid, blood, a blood fraction, or fine needle biopsy samples (e.g., surgical biopsy, fine needle biopsy, etc.), urine, peritoneal fluid, pleural fluid, and the like. Although the sample is often taken from a human subject (e.g., patient), the sample may be from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

The term “subject” herein refers to a human subject as well as a non-human subject such as a mammal, an invertebrate, a vertebrate, a fungus, a yeast, a bacterium, and a virus. Although the examples herein concern humans and the language is primarily directed to human concerns, the concepts disclosed herein are applicable to genomes from any plant or animal, and are useful in the fields of veterinary medicine, animal sciences, research laboratories and such.

The term “condition” or “medical condition” is used herein as a broad term that includes all diseases and disorders, but can include injuries and normal health situations, such as pregnancy, that might affect a person's health, benefit from medical assistance, or have implications for medical treatments.

As used herein, the term “cluster” or “clump” refers to a group of molecules, e.g., a group of DNA, or a group of signals. In some embodiments, the signals of a cluster are derived from different features. In some embodiments, a signal clump represents a physical region covered by one amplified oligonucleotide. Each signal clump could be ideally observed as several signals. Accordingly, duplicate signals could be detected from the same clump of signals. In some embodiments, a cluster or clump of signals can comprise one or more signals or spots that correspond to a particular feature. When used in connection with microarray devices or other molecular analytical devices, a cluster can comprise one or more signals that together occupy the physical region occupied by an amplified oligonucleotide (or other polynucleotide or polypeptide with a same or similar sequence). For example, where a feature is an amplified oligonucleotide, a cluster can be the physical region covered by one amplified oligonucleotide. In other embodiments, a cluster or clump of signals need not strictly correspond to a feature. For example, spurious noise signals may be included in a signal cluster but not necessarily be within the feature area. For example, a cluster of signals from four cycles of a sequencing reaction could comprise at least four signals.

The term “next generation sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “read” or “sequence read” (or sequencing reads) refer to a sequence obtained from a portion of a nucleic acid sample. A read may be represented by a string of nucleotides sequenced from any part or all of a nucleic acid molecule. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene. For example, a sequence read may be a short string of nucleotides (e.g., 20-150 bases) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

The term “sequencing depth,” as used herein, generally refers to the number of times a locus is covered by a sequence read aligned to the locus. The locus may be as small as a nucleotide, or as large as a chromosome arm, or as large as the entire genome. Sequencing depth can be expressed as 50×, 100×, etc., where “×” refers to the number of times a locus is covered with a sequence read. Sequencing depth can also be applied to multiple loci, or the whole genome, in which case x can refer to the mean number of times the loci or the haploid genome, or the whole genome, respectively, is sequenced. When a mean depth is quoted, the actual depth for different loci included in the dataset spans over a range of values. Ultra-deep sequencing can refer to at least 100× in sequencing depth.

The term “coverage” refers to the abundance of sequence tags mapped to a defined sequence. Coverage can be quantitatively indicated by sequence tag density (or count of sequence tags), sequence tag density ratio, normalized coverage amount, adjusted coverage values, etc. In some cases, “effective read coverage” of a chromosome is defined as the actual amount of bases covered by reads. Sequencing depth, which refers to the expected coverage of nucleotides by reads, is computed based on the assumption that reads are synthesized uniformly across chromosomes. In reality, read coverage across genomes is not uniform. Although a coverage of 10×, for example, means a nucleotide is covered 10 times on average, in certain parts of a genome, nucleotides are covered much more or much less. One factor that influences coverage is the ability of a read aligner to align reads to genomes. If a part of a genome is complex, e.g. having many repeats, aligners might have troubles aligning reads to that region, resulting in low coverage.

As used herein, the terms “aligned,” “alignment,” or “aligning” refer to the process of comparing a read or tag to a reference sequence and thereby determining the likelihood of the reference sequence contains the read sequence. If the reference sequence contains the read, the read may be mapped to the reference sequence or, in certain embodiments, to a particular location in the reference sequence. For example, the alignment of a read to the reference sequence for human chromosome 13 will tell the likelihood of the read is present in the reference sequence for chromosome 13. In some cases, an alignment additionally indicates a location where the read or tag maps to in the reference sequence. For example, if the reference sequence is the whole human genome sequence, an alignment may indicate that a read is present on chromosome 13, and may further indicate that the read is on a particular strand and/or site of chromosome 13. A “site” may be a unique position on a polynucleotide sequence or a reference genome (i.e. chromosome ID, chromosome position and orientation). In some embodiments, a site may provide a position for a residue, a sequence tag, or a segment on a sequence.

Aligned reads or tags are one or more sequences that are identified as a match in terms of the order of their nucleic acid molecules to a known sequence from a reference genome. Alignment can be done manually, although it is typically implemented by a computer algorithm, as it would be impossible to align reads in a reasonable time period for implementing the methods disclosed herein. The matching of a sequence read in aligning can be a 100% sequence match or less than 100% (non-perfect match).

Alignment may be performed by modifications and/or combinations of methods such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRIMP, SLIDER, SOAP, SOAP2, SOAP3 and SOAP3-dp, SOCS, SSAHA and SSAHA2, Stampy, STORM, Subread and Subjunc, Taipan, UGENE, VelociMapper, XpressAlign, and ZOOM.

The term “mapping” used herein refers to specifically assigning a sequence read to a larger sequence, e.g., a reference genome, by alignment.

A “genetic variation” or “genetic alteration” refers to a particular genotype present in certain individuals, and often a genetic variation is present in a statistically significant sub-population of individuals. The presence or absence of a genetic variance can be determined using a method or apparatus described herein. In certain embodiments, the presence or absence of one or more genetic variations is determined according to an outcome provided by methods and apparatuses described herein. In some embodiments, a genetic variation is a chromosome abnormality (e.g., aneuploidy), partial chromosome abnormality or mosaicism, each of which is described in greater detail herein. Non-limiting examples of genetic variations include one or more deletions (e.g., micro-deletions), duplications (e.g., micro-duplications), insertions, mutations, polymorphisms (e.g., single-nucleotide polymorphisms), fusions, repeats (e.g., short tandem repeats), distinct methylation sites, distinct methylation patterns, the like and combinations thereof. An insertion, repeat, deletion, duplication, mutation or polymorphism can be of any length, and in some embodiments, is about 1 base or base pair (bp) to about 250 megabases (Mb) in length. In some embodiments, an insertion, repeat, deletion, duplication, mutation or polymorphism is about 1 base or base pair (bp) to about 1,000 kilobases (kb) in length (e.g., about 10 bp, 50 bp, 100 bp, 500 bp, 1 kb, 5 kb, 10 kb, 50 kb, 100 kb, 500 kb, or 1000 kb in length).

A genetic variation is sometimes a deletion. In certain embodiments a deletion is a mutation (e.g., a genetic aberration) in which a part of a chromosome or a sequence of DNA is missing. A deletion is often the loss of genetic material. Any number of nucleotides can be deleted. A deletion can comprise the deletion of one or more entire chromosomes, a segment of a chromosome, an allele, a gene, an intron, an exon, any non-coding region, any coding region, a segment thereof or combination thereof. A deletion can comprise a microdeletion. A deletion can comprise the deletion of a single base.

A genetic variation is sometimes a genetic duplication. In certain embodiments a duplication is a mutation (e.g., a genetic aberration) in which a part of a chromosome or a sequence of DNA is copied and inserted back into the genome. In certain embodiments a genetic duplication (i.e. duplication) is any duplication of a region of DNA. In some embodiments a duplication is a nucleic acid sequence that is repeated, often in tandem, within a genome or chromosome. In some embodiments a duplication can comprise a copy of one or more entire chromosomes, a segment of a chromosome, an allele, a gene, an intron, an exon, any non-coding region, any coding region, segment thereof or combination thereof. A duplication can comprise a microduplication. A duplication sometimes comprises one or more copies of a duplicated nucleic acid. A duplication sometimes is characterized as a genetic region repeated one or more times (e.g., repeated 1, 2, 3, 4, 5, 6, 7, 8, 9 or 10 times). Duplications can range from small regions (thousands of base pairs) to whole chromosomes in some instances. Duplications frequently occur as the result of an error in homologous recombination or due to a retrotransposon event. Duplications have been associated with certain types of proliferative diseases. Duplications can be characterized using genomic microarrays or comparative genetic hybridization (CGH).

A genetic variation is sometimes an insertion. An insertion is sometimes the addition of one or more nucleotide base pairs into a nucleic acid sequence. An insertion is sometimes a microinsertion. In certain embodiments an insertion comprises the addition of a segment of a chromosome into a genome, chromosome, or segment thereof. In certain embodiments an insertion comprises the addition of an allele, a gene, an intron, an exon, any non-coding region, any coding region, segment thereof or combination thereof into a genome or segment thereof. In certain embodiments an insertion comprises the addition (i.e., insertion) of nucleic acid of unknown origin into a genome, chromosome, or segment thereof. In certain embodiments an insertion comprises the addition (i.e. insertion) of a single base.

A genetic variation sometimes includes copy number variations, i.e., variations in the number of copies of a nucleic acid sequence present in a test sample in comparison with the copy number of the nucleic acid sequence present in a reference sample. In certain embodiments, the nucleic acid sequence is 1 kb or larger. In some cases, the nucleic acid sequence is a whole chromosome or significant portion thereof. A copy number variant may refer to the sequence of nucleic acid in which copy-number differences are found by comparison of a nucleic acid sequence of interest in test sample with an expected level of the nucleic acid sequence of interest. For example, the level of the nucleic acid sequence of interest in the test sample is compared to that present in a qualified sample. Copy number variants/variations may include deletions, including microdeletions, insertions, including microinsertions, duplications, multiplications, and translocations. CNVs encompass chromosomal aneuploidies and partial aneuploidies.

As used herein, the term “array” may refer to a sequence of given size in the genome. In some examples, an array may comprise the total length of a VNTR. In some examples, an array may include all of the repeat copies of a VNTR. In some examples, an array may further comprise another target region.

As used herein, the term “consensus pattern motif (logo)” refers to the consensus sequence of the VNTR pattern describing the frequency at which different bases occur at each position.

As used herein, the term “copy number” refers to the number of times (e.g., 0, 1, 1.5, 2, 3.5, 5, etc.) the repeat unit is repeated for a given VNTR. The change in copy number for a VNTR can be represented as the difference in copy number relative to the reference (e.g., −1, 0, +1, +2, etc.).

As used herein, the term “copy number genotype” refers to the diploid genotype of the changes in the copy numbers of a given VNTR relative to the reference, reported as two copy number change alleles (e.g., 0/+1, −1/−1, etc.)

As used herein, the term “fragment size” refers to the length of the original nucleic acid sequence used to generate paired-end reads, calculated based on where those reads are mapped.

As used herein, the term “indels” refers to small insertions or deletions less than 50 base pairs in length in a nucleic acid sequence.

As used herein, the term “paired-end reads” or “paired end reads” refers to paired reads generated from sequencing the forward and reverse ends of a larger nucleic acid fragment. In some examples, the forward and reverse ends of a larger nucleic acid fragment may share the same name. The paired-end reads may be generated from paired end sequencing that obtains one read from each end of a nucleic acid fragment.

As used herein, the term “pattern” refers to the sequence of a repeat unit of the tandem repeat.

As used herein, the term “mate” or “mate of a read” refers to the pair of the read in question; i.e., the other read generated from the same nucleic acid fragment.

As used herein, the term “repeat unit” refers to the sequence of a single copy that is repeated multiple times in a VNTR.

As used herein, the term “single nucleotide variants” or “SNVs” refers to single base substitutions in a nucleic acid sequence.

As used herein, the term “small variant event” refers to a collection of adjacent SNVs or indels that occurs in the same haplotype of the VNTR array within a maximal distance of each other (for example, a maximal distance of 10 base-pairs).

As used herein, the term “spanning fragment” refers to a read fragment that spans the length of the entire VNTR array such that the left and right paired-end reads are on the left and right flanks of the VNTR.

As used herein, the term “structural variation” or “SV” refers to a large nucleic acid variant greater than 50 base pairs corresponding to either a duplication, deletion, insertion, inversion, or translocation.

As used herein, the term “tandem repeat” or “TR” refers to a nucleic acid sequence with a repeat unit of at least 10 base pairs, where the repeat unit is repeated at least 1.6 times with a similarity score of at least 1.7, consistent with the definitions in “Benson, Gary. ‘Tandem repeats finder: a program to analyze DNA sequences.’ Nucleic acids research 27.2 (1999): 573-580, the disclosures of which are incorporated herein by reference in their entirety.

As used herein, the term “variable number tandem repeat” or “VNTR” refers to a tandem repeat that has been observed to vary in the number of copies (e.g., duplicated copies or deleted copies) in the population of a species.

As used herein, the term “VNTR array” refers to the sequence covering the entire length of a VNTR. The VNTR array includes all of the copies of the repeat units.

In some cases, two haplotypes of a VNTR may comprise different numbers of copies of the repeat unit. In some cases, two haplotypes of the VNTR may comprise an identical number of copies of the repeat unit. The repeat units in each of the two haplotypes can include differentiating bases. A sequence of the repeat unit of one of the two haplotypes and a sequence of the repeat unit of the other one of the two haplotypes can be different at one or more differentiating positions; these sequences can have (or can have at least) 70%, 75%, 80%, 85%, 90%, 95%, 99%, or more, sequence identity. A sequence of the repeat unit of one of the two haplotypes and a sequence of the repeat unit of the other one of the two haplotypes can be identical in some examples.

Each haplotype of a VNTR can comprise a plurality of copies of a repeat unit. The repeat unit can be (or be at least or be more than) 6 bps, 7 bps, 8 bps, 9 bps, 10 bps, 11 bps, 12 bps, 13 bps, 14 bps, 15 bps, 16 bps, 17 bps, 18 bps, 19 bps, 20 bps, or more in length. The number of the plurality of copies can be (or be at least or be more than) 1.6, or more. The pathogenic copy number can be equal to, more than, or less than, the copy number in the reference sequence.

Two copies of a repeat unit of a haplotype can include differentiating bases. For example, sequences of two copies of the repeat unit of a haplotype can be different at one or more differentiating positions (e.g., 2, 3, 4, 5, 10, 20, or more, positions). The sequences of the two copies of the repeat unit of a haplotype may have (or may have at least) 70%, 75%, 80%, 85%, 90%, 95%, 99%, or more, sequence identity. Sequences of two copies of the repeat unit of a haplotype can be identical in some examples.

### Embodiments of Methods of Detecting and Genotyping VNTRs

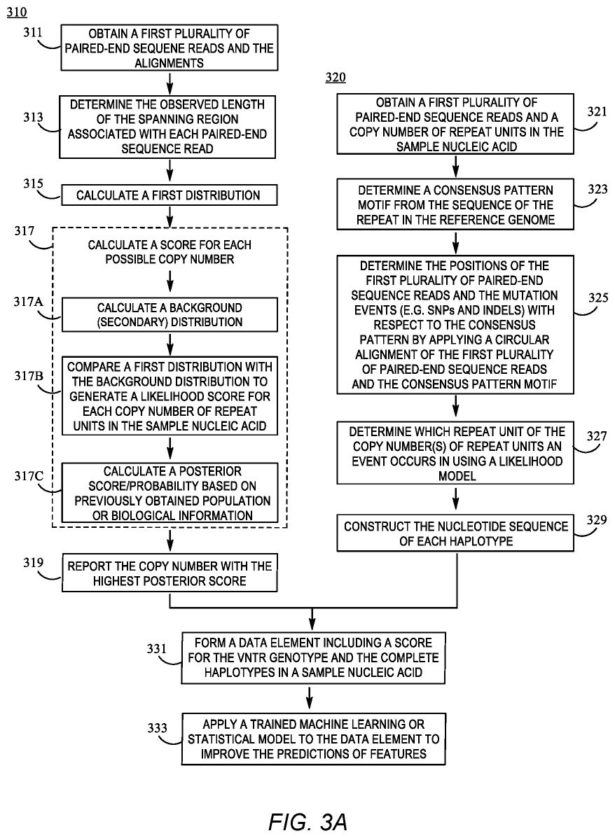

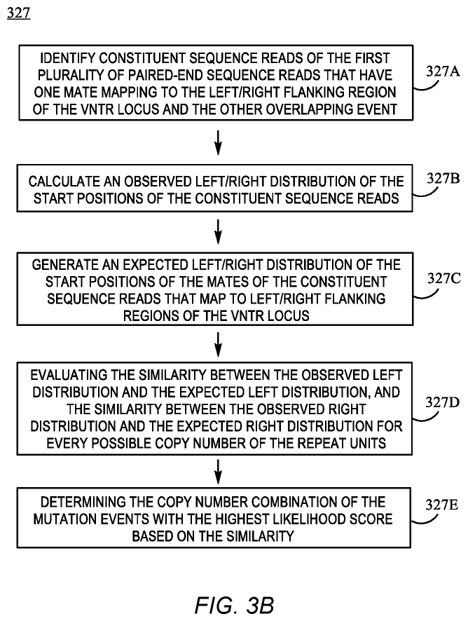

FIG. 3A and FIG. 3B are block diagrams that schematically illustrate exemplary methods of detecting and genotyping VNTRs. FIG. 3A illustrates a method 310 for determining a score for a copy number of repeat units in a sample nucleic acid, a method 320 for determining the nucleotide sequence of a sample nucleic acid having repeat units. FIG. 3B illustrates further details regarding a portion of the method 320. In some embodiments, results of the method 310 may be used in the method 320. In some embodiments, results of the method 310 and/or results of the method 320 may be used for predicting a feature of a subject.

As shown in FIG. 3A, the method 310 for determining a score for a copy number of repeat units in a sample nucleic acid may start from block 311, wherein a first plurality of paired-end sequence reads that each aligns and spans a VNTR array (i.e., aligns to the left and right flanks of the VNTR array) in a reference sequence and the alignments for the first plurality of paired-end sequence reads are both obtained. Next, the method 310 may proceed to block 313 wherein, based on the obtained alignments, the observed length of the spanning region associated with each paired-end sequence read is determined. Next, the method 310 may proceed to block 315 wherein a first distribution of the observed lengths of the spanning regions associated with the first plurality of paired-end sequence reads is calculated. Next, the method 310 may proceed to block 317 wherein a score for each possible copy number is calculated. Calculating a score for each possible copy number in block 317 may include step 317A wherein a background (secondary) distribution is calculated, step 317B wherein the first distribution is compared with the background distribution to generate a likelihood score for each copy number of repeat units in the sample nucleic acid, and step 317C wherein a posterior score/probability is calculated based on priorly obtained population or biological information. Then, the method 310 may proceed to block 319 to report the copy number with the highest posterior score.

The first plurality of paired-end sequence reads and the second plurality of paired-end sequence reads may be subsets of the paired-end sequence reads for the sample nucleic acid. The normalization sequences may be evolutionarily conserved regions in the reference sequence and/or regions in the reference sequence that do not comprise structural variation events. The alignments in block 311 may tolerate a degree of mismatch lower than a threshold.

In some embodiments, calculating a secondary distribution in step 317A may include generating for at least one possible copy number of repeat units, at least one secondary distribution of expected spanning region lengths based in part on the background distribution, the at least one possible copy number, the pattern of the VNTR locus, and copy number of the VNTR locus In some embodiments, comparing the first distribution with the background distribution in step 317B may include evaluating the similarity between the first distribution and the at least one secondary distribution. The at least one secondary distribution may be generated by statistical modeling in one embodiment. For example, the at least one secondary distribution may be generated by shifting the lengths of the mapped segments of background distribution by (N_{V}−N_{C})·S_{V}, wherein N_{C} is the at least one possible copy number, N_{V} is the copy number of repeat units in the VNTR locus, S_{V} is the length of a repeat unit in the VNTR locus. In some embodiments, evaluating the similarity between the first distribution and the at least one secondary distribution is by way of a statistical test.

In some embodiments, the method 310 may further include confirming that at least 40, at least 50, at least 60, at least 70, at least 80, at least 90 or at least 100 paired-end sequence reads align to spanning regions of the VNTR locus in the reference sequence. In some embodiments, the method 310 may further include confirming that the mean of the first distribution and the mean of the background distribution differs by at least 30, 40, 50, 60, 70, 80, 90 or 100 base pairs in length.

In some embodiments, a repeat unit in the VNTR locus is longer than about 5, 10, 20, 30, 40, 50, 60 or 70 base pairs in length. In some embodiments, the VNTR locus is about 300 base pairs to about 600 base pairs in length. In some embodiments, the VNTR locus is referred to as a macrosatellite or a minisatellite. In some embodiments, the macrosatellite has repeat patterns of longer than 100 base pairs in length. In some embodiments, the minisatellite has repeat patterns of about 10 base pairs to about 100 base pairs in length.

In some embodiments, each paired-end sequence read is about 100 base pairs to about 500 base pairs in length. In some embodiments, the paired-end sequence reads for the sample nucleic acid is generated by targeted sequencing, whole genome sequencing (WGS), or clinical WGS. In some embodiments, the paired-end sequence reads for the sample nucleic acid is generated by a next generation sequencing reaction. In some embodiments, each paired-end sequence read is obtained from a nucleic acid cluster on a solid substrate. In some embodiments, the nucleic acid cluster on the solid substrate is generated by a bridge amplification process.

In some embodiments, the sample nucleic acid is extracted from cells, a cell-free DNA sample, an amniotic fluid, a blood sample, a biopsy sample, or a combination thereof. In some embodiments, the sample nucleic acid is from a human subject and the reference sequence is a portion of a consensus human genome or transcriptome.

As shown in FIG. 3A, the method 320 for determining (or resolving or reconstructing) the nucleotide sequence of a sample nucleic acid having repeat units may start from block 321 wherein a first plurality of paired-end sequence reads that each aligns to a spanning region of a variable number tandem repeat (VNTR) locus in a reference sequence, and a copy number of repeat units in the sample nucleic acid is obtained. Next, the method 320 may proceed to block 323 to determine the consensus pattern motif from the sequence of the repeat in the reference genome, wherein the consensus pattern motif comprises a plurality of events of single nucleotide variants or indels. Next, the method 320 may proceed to block 325 to determine the positions of the first plurality of paired-end sequence reads and the mutation events (e.g., SNPs and indels) with respect to the consensus pattern by applying circular alignment on the first plurality of paired-end sequence reads and the consensus pattern motif. Then, the method 320 may proceed to block 327 to determine which repeat unit of the copy number(s) of repeat units an event occurs in, using a likelihood model. In some embodiments, the method 320 may proceed to block 328 to obtain the most likely copy number of repeat units from results of the method 310, which may be used as the copy number of repeat units in the sample nucleic acid. Then, the method 320 may proceed to block 329 to constructing the nucleotide sequence of each haplotype.

In some embodiments, the block 323 of determining the consensus pattern motif from the sequence of the repeat in the reference genome comprises remapping the first plurality of paired-end sequence reads to the VNTR locus by a circular alignment process. In some embodiments, the method 320 further includes obtaining the alignments for the first plurality of paired-end sequence reads; and determining, prior to the remapping, whether single nucleotide variants or indels occur in the first plurality of paired-end sequence reads based on the obtained alignments. For example, the method 320 may only proceed to block 323 if single nucleotide variants or indels do occur in the first plurality of paired-end sequence reads based on the obtained alignments.

In some embodiments, as shown in FIG. 3B, the block 327 shown in FIG. 3A of determining which repeat unit of the copy number of repeat units the event occurs in may include step 327A to identify constituent sequence reads of the first plurality of paired-end sequence reads that have one mate mapping to the left/right flanking region of the VNTR locus and the other overlapping the event. After step 327A, the block 327 may proceed to step 327B to calculate an observed left/right distribution of the start positions of the constituent sequence reads (based on the results from step 327A). After step 327C, the block 327 may proceed to step 327C to generate, assuming that the event occurs in the j′th repeat unit of the copy number of repeat units, an expected left distribution of the start positions of the mates of the constituent sequence reads that map to a left flanking region of the VNTR locus, and an expected right distribution of the start positions of the mates of the constituent sequence reads that map to a right flanking region of the VNTR locus. After step 327C, the block 327 may proceed to step 327D to evaluate the similarity between the observed left distribution and the expected left distribution, and the similarity between the observed right distribution and the expected right distribution, for every possible copy number of the repeat units. After step 327D, the block 327 may proceed to step 327E to determining the copy number combination of the mutation events with the highest likelihood score based on the similarity calculated above. In some embodiments, evaluating the similarity between the distributions is by way of a statistical. In some embodiments, the expected left distribution and the expected right distribution are generated by statistical modeling. For example, the expected left distribution may be a distribution of the lengths of normalization sequences in the reference sequence that are greater than the distance from the event to the left flank region of the VNTR, given that the event occurs in the j′th repeat unit. For example, the expected right distribution may be a distribution of the lengths of normalization sequences in the reference sequence that are greater than the distance from the event to the right flank region of the VNTR, given that the event occurs in the j′th repeat unit. The normalization sequences may be evolutionarily conserved regions in the reference sequence or regions in the reference sequence that do not comprise structural variation events.

As shown in FIG. 3A, the predicting a state/feature (e.g., an identity or a disease) of a subject may start from block 331 to form a data element (a unit of data, e.g., a vector or a tensor) including a score for the VNTR genotype and the complete haplotypes in a sample nucleic acid. After forming the data element in block 331, the method may proceed to block 333 to apply a trained machine learning or statistical model to the data element to improve the predictions of features.

In some embodiments, the data element may include the most likely copy number of repeat units in the sample nucleic acid, a distance between the VNTR locus and a gene in the genome of the subject, the length of a repeat unit in the VNTR locus, the GC content of the VNTR locus, the degree of mismatch in the alignments, and/or the probability that the VNTR locus in the reference sequence has mutated. In some embodiments, the data element may include the resolved nucleotide sequence of the sample nucleic acid having repeat units. The score for a copy number of repeat units or the most likely copy number of repeat units in the sample nucleic acid may be obtained from results of the method 310. The resolved nucleotide sequence of the sample nucleic acid having repeat units may be obtained from results of the method 320.

In some embodiments, tandem repeat features that can be utilized in predicting the state/feature of the subject include tandem repeat pattern size, copy number, GC content, an alignment score determining how well the repeat copies align to each other (as defined in “Benson, Gary. ‘Tandem repeats finder: a program to analyze DNA sequences.’ Nucleic acids research 27.2 (1999): 573-580”, the disclosures of which are incorporated herein by reference in their entirety), annotation (e.g., how close to genes the tandem repeats are), and how likely the tandem repeats mutate across humans. Scoring of the disclosed methods may also be utilized. For example, the statistical model outputs certainty measures (similar to likelihood probability), which may also be input to the machine learning model.

### Example Process I: Estimating the Copy Number Genotype

### Fragment Size Distribution Modeling

This exemplary method uses the distribution of fragment sizes to determine the most likely repeat copy number. In some embodiments, the most likely copy number is the one whose fragment size distribution as modeled by the background normalization regions most closely matches the observed fragment size distribution.

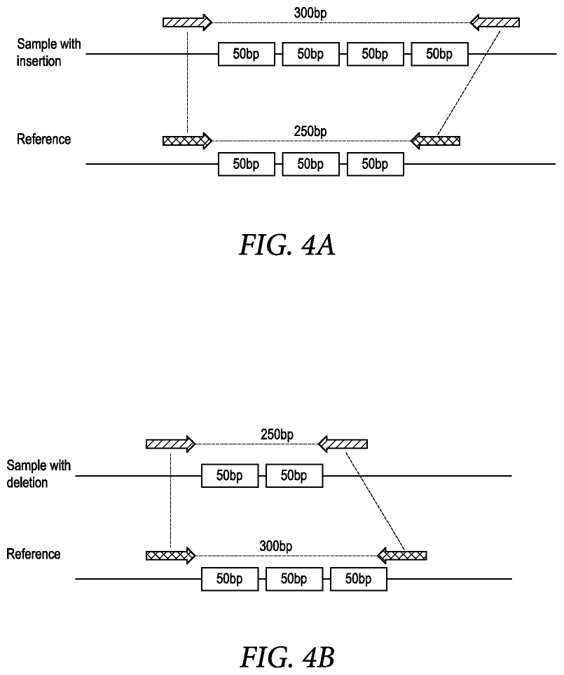

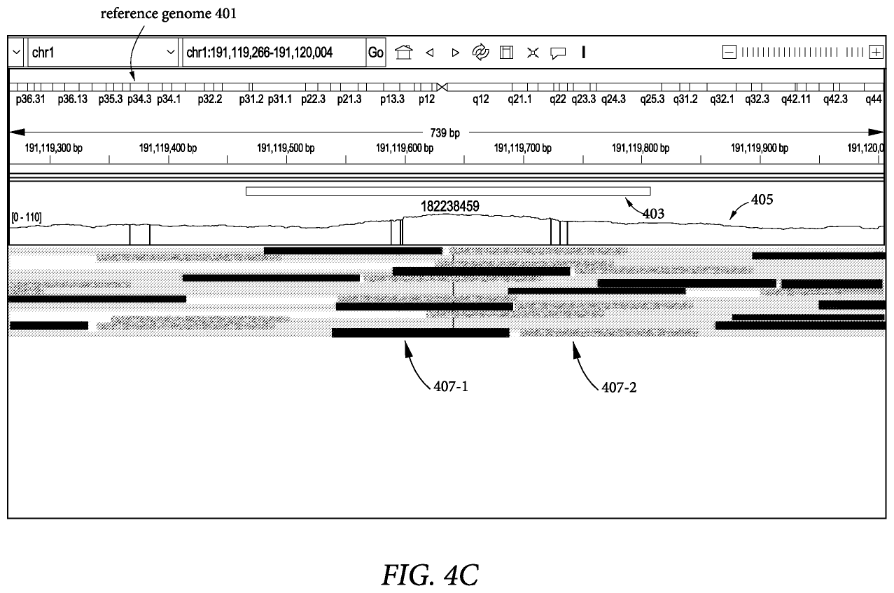

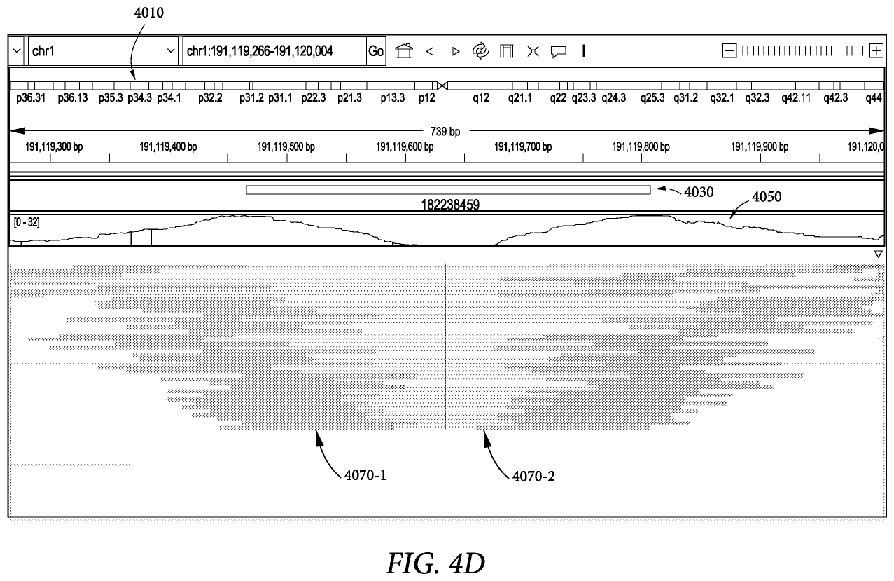

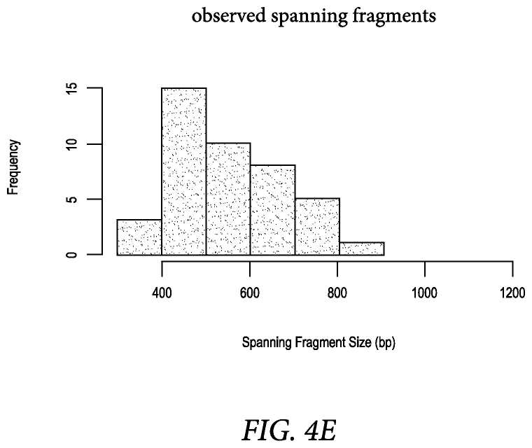

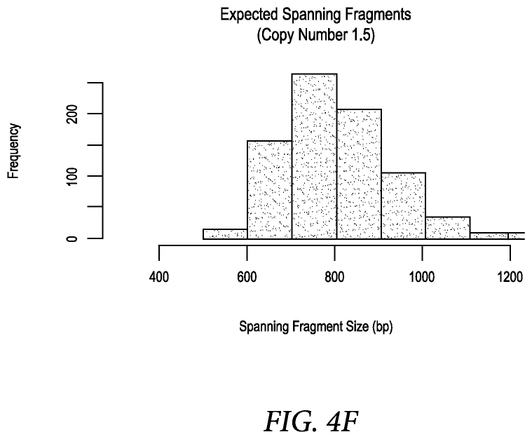

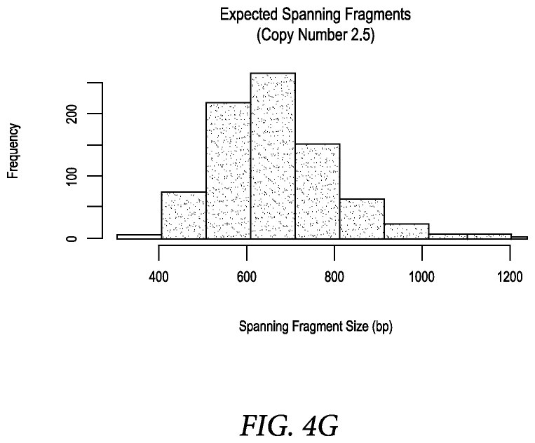

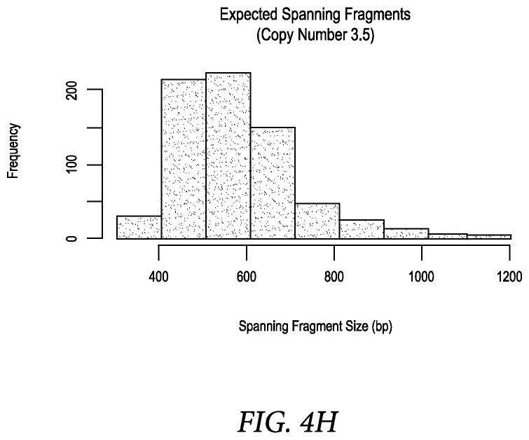

First, the background distribution may be considered. For example, a number of normalization regions have been chosen which are known to be evolutionarily conserved and thus are not expected to have Structural Variation (SV) events. Each region is 2000 base-pairs long, for example. The fragment sizes of the reads that map to these normalization regions provide a baseline for the expected fragment size distribution for a possible homozygous genotype in a sample-specific way. To do so, the fragments spanning a corresponding array size over the normalization regions may be extracted. This corresponding array size is equal in length to the VNTR genotype being considered. For example, a single copy deletion would correspond to the VNTR array size minus the pattern size and a single copy insertion would correspond to the VNTR array size plus the pattern size. These extracted fragments may be referred to as the baseline fragments. Then the expected fragment size distributions for a single copy deletion (−1), a double copy deletion (−2), etc. may be modeled by incrementing the baseline fragment sizes by the corresponding multiple of pattern size lengths (for example, +1*pattern size, +2*pattern size, etc.). Similarly, the expected fragment size distributions for a single copy insertion (+1), a double copy insertion (+2), etc. may be modeled by decrementing the baseline fragment sizes by the corresponding multiple of pattern size lengths. When there is a repeat copy insertion in a VNTR, spanning fragments become shorter; and when there is a deletion, the fragments become larger (see FIG. 4A and FIG. 4B which illustrate that an exemplary distribution of the spanning fragment sizes varies with single copy insertions and deletions in VNTRs. FIG. 4A shows a sample polynucleotide having a single copy insertion compared to the reference polynucleotide. FIG. 4B shows a sample polynucleotide having a single copy deletion compared to the reference polynucleotide.). Additionally, to calculate a possible heterozygous genotype, a 50/50 mixture of the corresponding homozygous baseline fragment sizes may be used. This can be extended for any ploidy level.

Next, for each VNTR region in the sample, all spanning read fragments (i.e., reads whose left read pair maps to the left flank of the VNTR and reads whose right read pair maps to the right flank of the VNTR) may be considered. There is a minimum sequence length of the left flank of the VNTR that the left read pair must align to and vice versa for the right side (the default minimum sequence length may be chosen as 16 bp, for example). The fragment size distributions of the spanning read fragments may be determined, and a likelihood test comparing the observed distribution with the expected distributions may be used to find the most likely copy number for each VNTR.

### Likelihood Modeling of Copy Number

The copy number of each VNTR may be determined by performing a non-parametric likelihood test on the observed fragment size distributions against the expected fragment size distributions for all possible copy number genotypes.

For each VNTR, the observed fragment size distribution may be obtained from the set of flanking fragments for that VNTR, and the expected distributions for each copy number genotype may be obtained from the background normalization regions. Every copy number genotype is considered up to an array size of the maximum fragment size (e.g., 1000 base-pairs): homozygous reference (0/0), heterozygous single deletion (0/−1), homozygous single deletion (−1/−1), heterozygous single insertion (0/+1), etc. Then, the likelihood of each possible genotype may be calculated as P(genotype|fragments)≈P(fragments|genotype)×P(genotype); where P(genotype) is the prior probability of that genotype. This prior probability can be calculated based on population information of biological hypothesis. P(fragments genotype) can be calculated by different approaches such as by considering the probability of each fragment: ΠP(each fragment|background distribution of the genotype).

### Genotyping and Scoring

Next, the chosen copy number genotype may be used to generate an SV variant call. The copy number genotype may be converted into a deletion or insertion SV call, and then may be scored based on additional methods. These methods could include simulating an assembly contig based on the variant call, simulating the alignment of the assembly contig to the reference, finding supporting reads, etc.

### Illustration of the Example Process I

A VNTR with the TRDB ID number 182238459 has pattern length 135 and 2.5 copies in the hg38 reference genome. In the HG002 (NA24385) sample, this VNTR has 3.5 copies on each chromosome. How the Example Process I detects this homozygous gain of one copy (+1/+1) is illustrated as follows.

FIG. 4C, FIG. 4D and FIG. 4E illustrate exemplary results of extraction of spanning fragments for a target VNTR region and modeling of the spanning fragment size distribution. FIG. 4C is a plot that shows all the reads that align to the VNTR region. The plot includes the reference genome 401, the bar 403 representing the VNTR region on the reference genome 401, the histogram plot 405 representing the depth of aligned reads to this VNTR region, and the lines 407 representing paired-end reads that align to the VNTR region. From all the reads in FIG. 4C, the paired-ends that span the VNTR are extracted and shown in FIG. 4D. FIG. 4D is a plot that shows the extracted spanning fragments. The plot includes the reference genome 4010, the bar 4030 representing the VNTR region on the reference genome 4010, the histogram plot 4050 representing the depth of aligned reads to this VNTR region, and the lines 4070-1 and 4070-2 representing two particular paired-end reads that align to the VNTR region. The size of the spanning fragments from FIG. 4D are then calculated, and the primary distribution of the sizes of the observed spanning fragments is shown in the histogram of FIG. 4E.

To get the expected copy number, first, one can calculate the corresponding array size as copy number * pattern size. For example, for this VNTR with pattern size 135 base-pairs and reference copy number 2.5, the corresponding reference VNTR array size is 341 base-pairs. A loss of one copy (i.e., having 1.5 copies) would correspond to an array size of 206 bp, no change (i.e., having 2.5 copies) would correspond to an array size of 341 by (same as the reference VNTR array size), and a gain of one copy (i.e., having 3.5 copies) would correspond to an array of size 476 bp. Then, over a list of normalization/background regions which are highly conserved (e.g., less than 1% variation among primates), one can extract spanning fragments for each of these array sizes. The list of normalization/background regions used in the disclosed method may be found in “Siepel A, Bejerano G, Pedersen J S, Hinrichs A S, Hou M, Rosenbloom K, Clawson H, Spieth J, Hillier L W, Richards S, et al. ‘Evolutionarily conserved elements in vertebrate, insect, worm, and yeast genomes.’ Genome Res. 2005 August; 15 (8): 1034-50”, the disclosures of which are incorporated herein by reference in their entirety. Then, the sizes of such extracted fragment are calculated, and the distribution of such extracted fragment sizes represent a “background distribution”. Next, one can add the opposite change to each size in the extracted fragment sizes, i.e., adding (reference VNTR copy number-possible copy number)*pattern size to each size in the extracted fragment sizes. This amount would be +135 for the loss of one copy (1.5 copies), 0 for no change (2.5 copies), and −135 for the insertion of one copy (3.5 copies). The secondary distributions are then calculated similar to the primary distribution.

FIG. 4F, FIG. 4G and FIG. 4H illustrate exemplary results of calculating secondary distributions for several possible copy numbers based on a set of candidate/normalization/background regions. FIG. 4F shows the expected fragment size distribution for copy number 1.5 (i.e., loss of one copy). The fragments spanning a region having a size corresponding to the loss of one copy (i.e., array size−one pattern=341 bp−135 bp=206 bp) would result in a distribution that is shifted by 135 base-pairs to the right compared to the background distribution. FIG. 4G shows the expected fragment size distribution for copy number 2.5 (i.e., no change). The fragments spanning a region that is the same as the reference VNTR (i.e., array size=341 bp) would result in a distribution that is not shifted compared to the background distribution. FIG. 4H shows the expected fragment size distribution for copy number 3.5 (i.e., gain of one copy). The fragments spanning a region having a size corresponding to gain of one copy (i.e., array size+one pattern=341 bp+135 bp=476 bp) would result in a distribution that is shifted by −135 bp to the left compared to the background distribution.

### Scoring of Copy Number Genotypes

Table 1 shows results from calculating the score for several possible copy numbers. The spanning fragments sizes of the VNTR may be used to calculate the primary distribution. For a given copy number, the expected spanning fragment sizes extracted from candidate/normalization/background regions (as explained in FIG. 4F, FIG. 4G and FIG. 4H.) are used to calculate the secondary distribution. The similarity of the primary and secondary distributions may be evaluated by calculating the product of the probability of observing each fragment size from the primary distribution, against the secondary distribution. Other tests would be applied to calculate the probability of the primary distribution to the secondary distribution. This probability score may be adjusted with prior information from the existing data on common VNTRs in the population and/or biological models of VNTR variation. The final score may be calculated as P(primary distribution and secondary distribution are the same)*Prior from population data. This score correlates to the posterior probability of the genotype. P(genotype|distribution)=P(distribution|genotype)*P(genotype)/P(distribution)∝P(distribution|genotype)*P(genotype).

TABLE 1

| | | P(fragments \| | Prior from | |
|---|---|---|---|---|
| Genotype | Event | genotype) | population data | Score |
| −1/−1 | Homozygous | 6E−21 | 0.15 | 9E−22 |
| | single | | | |
| | deletion | | | |
| 0/−1 | Heterozygous | 1E−12 | 0.25 | 3E13 |
| | single | | | |
| | deletion | | | |
| 0/0 | Reference | 3E06 | 0.35 | 1E06 |
| | (no change) | | | |
| 0/+1 | Heterozygous | 1E02 | 0.20 | 2E−03 |
| | single | | | |
| | insertion | | | |
| +1/+1 | Homozygous | 7E01 | 0.05 | 4E02 |
| | single | | | |
| | insertion | | | |

For example, the genotype corresponding to a homozygous single insertion (+1/+1) has the highest score with 0.04.

### Determining the Full Target VNTR Array Sequence

If no SNVs or indels occur in the target/unknown VNTR array of the sample nucleic acid, then the full sequence of the target/unknown VNTR array may be simply determined by the repeat unit pattern of the reference VNTR region and the copy number of repeat units. The target/unknown VNTR array sequence can be reconstructed by simply generating a sequence with the appropriate number of repeat units based on the copy number genotype (e.g., determined by Example Process I). The output in that case may be the copy number genotype and the reconstructed VNTR array sequence.

On the other hand, if there are SNVs and indels in the target/unknown VNTR array of the sample nucleic acid, then an additional haplotype resolution process may be taken to assign the SNVs and indels to the correct repeat units and haplotypes. An example process for resolving these haplotypes is described in Example Process II below.

### Example Process II: Resolving the Full Haplotype Sequence (VNTR Assembly)

First, a circular alignment algorithm (based on “Benson, Gary. ‘Tandem cyclic alignment.’ Discrete applied mathematics 146.2 (2005): 124-133”, the disclosures of which are incorporated herein by reference in their entirety) may be utilized to map the reads to the VNTR pattern in a repeat-aware manner. From the output of that alignment, all of the small variant events of the VNTR may be obtained as well as the reads that overlap those events. The mates of those overlapping reads that align to the flanking regions of the VNTR are considered, and their fragment size distributions may be modeled. Those fragment size distributions may be compared to the distributions expected for events occurring at different copies of the VNTR (e.g., whether the event occurs on the first copy, on the second copy, etc.). The distribution that best matches the observed fragment size distribution may be determined, and the event may be assigned to that repeat copy. Then, once all of the events are assigned to repeat copies, the full VNTR sequence haplotypes may be reconstructed from the copy number and the Pattern Motif Logo, inputting the events into the assigned repeat copies.

### Circular Alignment

The positions of known reference VNTRs and their sequences are obtained. All of the reads that map to those positions may be extracted as well as the mates of the reads. Then, each reference VNTR may be considered separately.

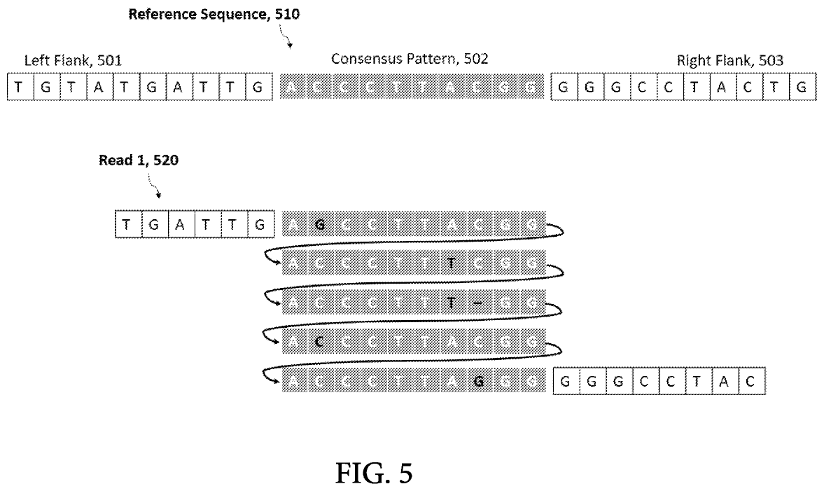

For each reference VNTR, a circular alignment procedure, based on “Benson, Gary. ‘Tandem cyclic alignment.’ Discrete applied mathematics 146.2 (2005): 124-133”, the disclosures of which are incorporated herein by reference in their entirety, may be performed to remap the reads to the reference VNTR array. Specifically, the reads may be aligned to the pattern consensus derived from the reference VNTR array. The reference VNTR array may be represented as a graph sequence as shown in FIG. 5 to be a part of the reference sequence 510 (with the left flank 501, the consensus pattern 502 and the right flank 503 consecutively). When a read 520 (“Read 1”) is circular aligned to the graph and reaches the end of the reference VNTR consensus pattern, it can either continue on into the flanking sequence or wrap-around (see the thick arrows in FIG. 5) to the beginning of the pattern with no penalty in score; the best/highest scoring alignment will be chosen. In some embodiments, the circular alignment also uses a scoring scheme that alters the alignment match scores based on the frequency at which different bases occur in the reference consensus pattern. From this circular alignment, the start and end positions of all reads with respect to the reference VNTR pattern may be obtained. The consensus pattern motif of the read sequences in the reference VNTR may also be constructed. The consensus pattern motif describes all of the SNVs and indels present in the reads to be assigned to specific repeat copies in the subsequent process.

### Event Definition and Fragment Size Distribution Modeling

All of the SNVs and indels in the Pattern Motif Logo may be represented as events. SNVs and indels that occur within a distance based on the read length of each other on the same haplotype are compiled into the same event. Events are defined by starting with a SNV or indel in the pattern, collecting reads that overlap the small variant, and then determining if those reads contain another SNV or indel within the event distance on either end. If so, those small variants will be included into the same event and the process will iterate on those variants. The steps may repeat until all SNVs and indels are included in the set of events.

The objective is to find which repeat copy each event begins on (i.e., where the first small variant of the event occurs). This may be done by looking at where the mates of the reads overlapping the event map to on the left and right flanking regions of the reference VNTR. For each event, all overlapping reads that support the event are found, as are the mates and fragment sizes of those reads. If the mate maps to the left flanking region of the reference VNTR, it is included in a list of left fragments and vice versa for mates that map to right fragments. The distribution may be calculated for starting positions of all of the mapped left fragments, labeled the observed left distribution. A similar procedure may be performed for the right fragments to construct the observed right distribution.

### Likelihood Modeling of Event Location

The observed left and right distributions may be compared to the expected distributions assuming the event begins on the first repeat copy, the second repeat copy. etc. The expected distribution of the repeat copy location that best matches the observed distribution may be chosen as the repeat copy for which the event begins.

For each repeat copy (e.g., based on the VNTR copy number calculated in Example Process I), the expected distribution of the flanking fragments may be simulated by considering the fragment size distributions of the normalization/background regions and removing fragments from the distribution that are shorter than the distance from the event to the VNTR flank. The expected distance from the event to each VNTR flank may be calculated based on the distance of the start of the event to each end of the pattern, given the repeat copy that the event is simulated for. Fragments shorter than this expected distance may be removed because they would not be expected to appear in the observed left and right distributions of fragments that map to the flanking regions. For the rare case that the same event occurs on several repeat copies, the expected distribution may be simulated for all possible combinations of repeat copies rather than expecting the event to only occur on a single repeat copy. Further information can be used in this scenario includes the presence of read pairs where both mates contain the event.

After obtaining the expected distributions, a procedure similar to that described in Example Process I may be used to determine the distribution that best matches the observed one. The likelihoods of the left and right distributions with the same event locations will be combined into one score (e.g., multiplied). The expected distribution with the highest score may be chosen, and the location of the event may be assigned to the corresponding repeat copy.

Once all of the events have been assigned to repeat copies, the fully resolved haplotypes of the VNTR array sequence may be constructed by filling in those SNVs and indels. The output of the algorithm may be the VNTR copy number and the fully resolved haplotype array sequences.

### Example Process

Circular align the reads to the consensus pattern and extract all events for each event:
leftDistance=distance of event from left flank
rightDistance=distance of event from right flank
leftFragments=read 1 is in left flank && read 2 supports event
rightFragments=read 2 is in right flank && read 1 supports event
leftObserved=start positions of read 1 in leftFragments
rightObserved=start positions of read 2 in rightFragments
for C in 1 to copy Number:

$$leftExpected = normalization region fragment sizes which are greater than leftDistance$$

$$rightExpected = normalization region fragment sizes which are greater than right Distance$$

$$fit [ C ] = P ( leftObserved \vert leftExpected ) ⋆ P ( rightObserved \vert rightExpected$$

$$predictedCopy = which . max ( fit )$$

$$return predictCopy$$

### Illustration of the Example Process II (Haplotype Resolver)

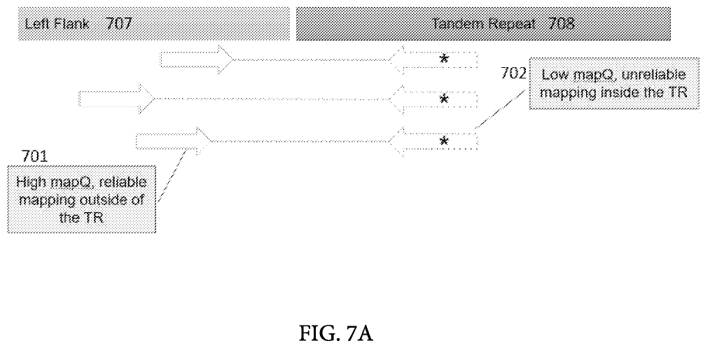

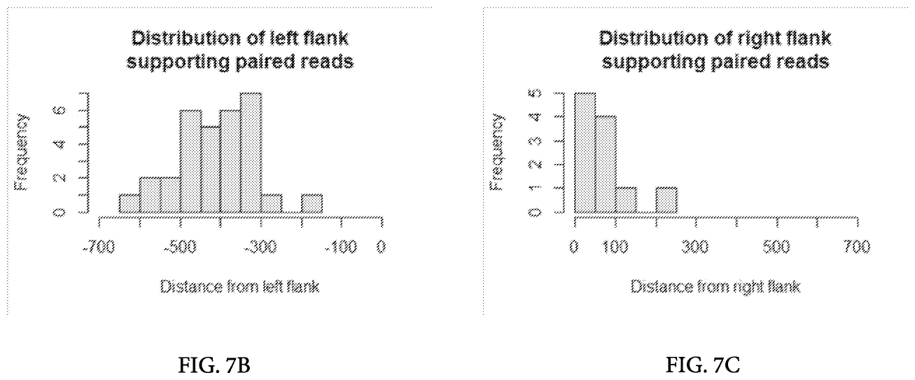

Assume a TR has pattern size 100 bp and genotype ⅔ (seven copies on each chromosome, 14 copies total), then the total array size is 200 bp and 300 bp on each paternal chromosome. If the sequencing read length is 100 bp, and fragment coverage is 100× and read coverage is 50×, one can apply circular alignment and identify a SNP at position 10 of the consensus pattern given that its event frequency within the read patterns (e.g., allele frequency) is 40%. For example, an event of T>C with frequency 40% means that it occurred in 40% of the repeats in the reads.
1. This means there are 0.40*5=2 different copies containing this SNP. These six copies could be any of the following combinations:
   One homozygous copy; or
   Two heterozygous copies
2. Since there are total 5 copies, total choose (5,2)=10 combinations must be tested. For example: {(A1,A2), (A1,B1), (A1,B2), (A1,B3), (A2,B1), (A2,B2), (A2,B3), (B1,B2), (B1,B3), (B2,B3)} where A is the first paternal chromosome and B is the second chromosome. Numbers 1 to 3 indicate the copy number. Note that there are two copies on A and three copies on B (genotype is 2/3).
3. For this VNTR one can extract read pairs with one end spanning the left or right flank, and one end supporting the SNP. For example, FIG. 7A schematically illustrates the alignment of these reads for the left flank (707). The first pair (701) of the read has a high mapping quality (mapQ) score due to reliable mapping outside of the TR (708). The second pair (702) of the read has a low mapping quality (mapQ) score due to unreliable mapping inside the TR. The star on the second pair (702) of the read indicates that the SNP has been detected in those reads. The scenario is symmetrical for the right flank.
4. Then one can draw the distribution of start positions of these reads, as shown in FIG. 7B and FIG. 7C. FIG. 7B shows the frequency of the paired reads that support the left flank as a function of the reads' distance from the left flank. FIG. 7C shows the frequency of the paired reads that support the right flank as a function of the reads' distance from the right flank.
5. One can compare this distribution to the background distribution of the 10 different combinations. This method could detect the correct combination of copy numbers 98/100 times.

### Samples

In some embodiments, the sample comprises or consists of a purified or isolated polynucleotide derived from a tissue sample, a biological fluid sample, a cell sample, and the like. Suitable biological fluid samples include, but are not limited to blood, plasma, serum, sweat, tears, sputum, urine, sputum, ear flow, lymph, saliva, cerebrospinal fluid, ravages, bone marrow suspension, vaginal flow, trans-cervical lavage, brain fluid, ascites, milk, secretions of the respiratory, intestinal and genitourinary tracts, amniotic fluid, milk, and leukophoresis samples. In some embodiments, the sample is a sample that is easily obtainable by non-invasive procedures, e.g., blood, plasma, serum, sweat, tears, sputum, urine, sputum, ear flow, saliva or feces. In certain embodiments the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In certain embodiments, samples can be obtained from sources, including, but not limited to, samples from different individuals, samples from different developmental stages of the same or different individuals, samples from different diseased individuals (e.g., individuals with cancer or suspected of having a genetic disorder), normal individuals, samples obtained at different stages of a disease in an individual, samples obtained from an individual subjected to different treatments for a disease, samples from individuals subjected to different environmental factors, samples from individuals with predisposition to a pathology, samples individuals with exposure to an infectious disease agent, and the like.

In one illustrative, but non-limiting embodiment, the sample is a maternal sample that is obtained from a pregnant female, for example a pregnant woman. The maternal sample can be a tissue sample, a biological fluid sample, or a cell sample. In another illustrative, but non-limiting embodiment, the maternal sample is a mixture of two or more biological samples, e.g., the biological sample can comprise two or more of a biological fluid sample, a tissue sample, and a cell culture sample.

In certain embodiments samples can also be obtained from in vitro cultured tissues, cells, or other polynucleotide-containing sources. The cultured samples can be taken from sources including, but not limited to, cultures (e.g., tissue or cells) maintained in different media and conditions (e.g., pH, pressure, or temperature), cultures (e.g., tissue or cells) maintained for different periods of length, cultures (e.g., tissue or cells) treated with different factors or reagents (e.g., a drug candidate, or a modulator), or cultures of different types of tissue and/or cells.

In some embodiments, the use of the disclosed sequencing technology does not involve the preparation of sequencing libraries. In other embodiments, the sequencing technology contemplated herein involve the preparation of sequencing libraries. In one illustrative approach, sequencing library preparation involves the production of a random collection of adapter-modified DNA fragments (e.g., polynucleotides) that are ready to be sequenced.

Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain embodiments, the polynucleotides may originate in single-stranded form (e.g., ssDNA, RNA, etc.) and have been converted to dsDNA form. By way of illustration, in certain embodiments, single stranded mRNA molecules may be copied into double-stranded cDNAs suitable for use in preparing a sequencing library. The precise sequence of the primary polynucleotide molecules is generally not material to the method of library preparation, and may be known or unknown. In one embodiment, the polynucleotide molecules are DNA molecules. More particularly, in certain embodiments, the polynucleotide molecules represent the entire genetic complement of an organism or substantially the entire genetic complement of an organism, and are genomic DNA molecules (e.g., cellular DNA, cell free DNA (cfDNA), etc.), that typically include both intron sequence and exon sequence (coding sequence), as well as non-coding regulatory sequences such as promoter and enhancer sequences. In certain embodiments, the primary polynucleotide molecules comprise human genomic DNA molecules, e.g., cfDNA molecules present in peripheral blood of a pregnant subject.

Methods of isolating nucleic acids from biological sources may differ depending upon the nature of the source. One of skill in the art can readily isolate nucleic acids from a source as needed for the method described herein. In some instances, it can be advantageous to fragment large nucleic acid molecules (e.g. cellular genomic DNA) in the nucleic acid sample to obtain polynucleotides in the desired size range. Fragmentation can be random, or it can be specific, as achieved, for example, using restriction endonuclease digestion. Methods for random fragmentation may include, for example, limited DNase digestion, alkali treatment and physical shearing. Fragmentation can also be achieved by any of a number of methods known to those of skill in the art. For example, fragmentation can be achieved by mechanical means including, but not limited to nebulization, sonication and hydroshear.

In some embodiments, sample nucleic acids are obtained from as cfDNA, which is not subjected to fragmentation. For example, cfDNA, typically exists as fragments of less than about 300 base pairs and consequently, fragmentation is not typically necessary for generating a sequencing library using cfDNA samples.

Typically, whether polynucleotides are forcibly fragmented (e.g., fragmented in vitro), or naturally exist as fragments, they are converted to blunt-ended DNA having 5′-phosphates and 3′-hydroxyl. Protocols for sequencing may instruct users to end-repair sample DNA, to purify the end-repaired products prior to dA-tailing, and to purify the dA-tailing products prior to the adaptor-ligating steps of the library preparation.

In various embodiments, verification of the integrity of the samples and sample tracking can be accomplished by sequencing mixtures of sample genomic nucleic acids, e.g., cfDNA, and accompanying marker nucleic acids that have been introduced into the samples, e.g., prior to processing.

### Embodiments of Sequencing Systems

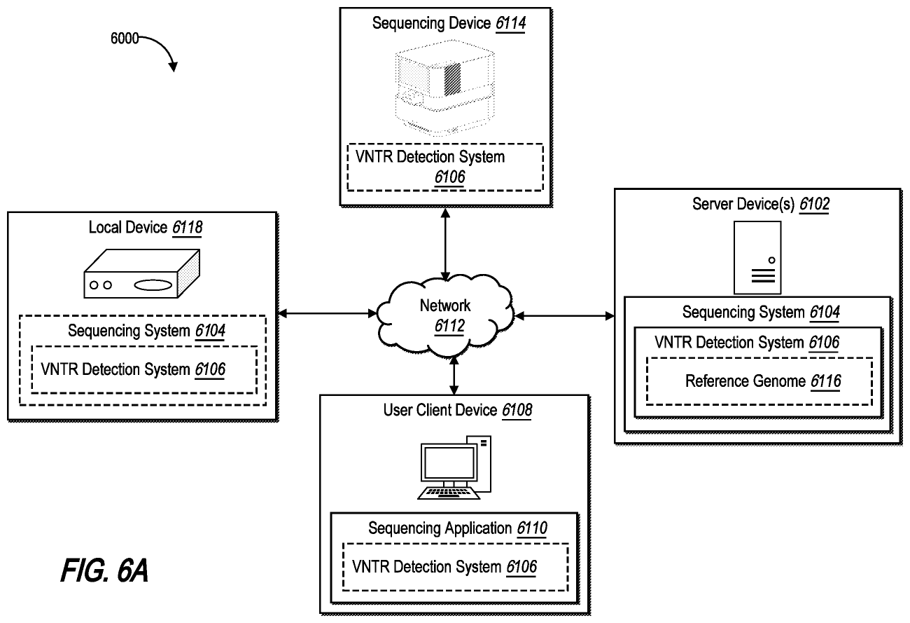

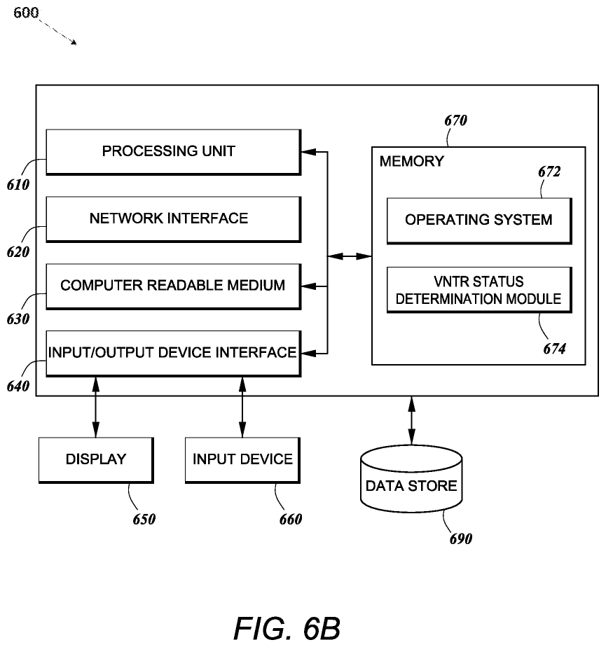

FIG. 6A is a block diagram of an exemplary sequencing system 6000 that may be used to perform the disclosed methods, such as method 3100 and/or, method 3200. For example, the sequencing system 6000 can be configured to determine a score for a copy number of repeat units in a sample nucleic acid. The illustrative sequencing system 6000 may include a nucleic acid sequencer 6001, a non-transitory memory 6003 configured to store executable instructions, and a hardware processor 6005 in communication with the nucleic acid sequencer 6001 and the non-transitory memory 6003. The hardware processor 6005 may be programmed by the executable instructions to perform the methods disclosed herein.

In some embodiments, the non-transitory memory 6003 is configured to store the reference sequence. In some embodiments, the hardware processor 6005 is configured to obtain the reference sequence from an external database. In some embodiments, the hardware processor 6005 is configured to receive paired-end sequence reads from the nucleic acid sequencer 6001. In some embodiments, the hardware processor 6005 is configured to control the nucleic acid sequencer 6001 to perform sequencing of the sample nucleic acid. In some embodiments, the hardware processor 6005 is configured to control the nucleic acid sequencer 6001 to perform additional sequencing of the sample nucleic acid based on the determined score for the most likely copy number of repeat units in the sample nucleic acid 6001. In some embodiments, the hardware processor 6005 is configured to output, on a display, the most likely copy number of repeat units in the sample nucleic acid and/or an associated score.

FIG. 6B is a block diagram of an exemplary computing device 600 that may be used in connection with the illustrative sequencing system 6000 of FIG. 6A. The computing device 600 may be configured to determine a VNTR status, such as genotyping a VNTR. The general architecture of the computing device 600 depicted in FIG. 6B includes an arrangement of computer hardware and software components. The computing device 600 may include many more (or fewer) elements than those shown in FIG. 6B. It is not necessary, however, that all of these generally conventional elements be shown in order to provide an enabling disclosure. As illustrated, the computing device 600 includes a processing unit 610, a network interface 620, a computer readable medium drive 630, an input/output device interface 640, a display 650, and an input device 660, all of which may communicate with one another by way of a communication bus. The network interface 620 may provide connectivity to one or more networks or computing systems. The processing unit 610 may thus receive information and instructions from other computing systems or services via a network. The processing unit 610 may also communicate to and from memory 670 and further provide output information for an optional display 650 via the input/output device interface 640. The input/output device interface 640 may also accept input from the optional input device 660, such as a keyboard, mouse, digital pen, microphone, touch screen, gesture recognition system, voice recognition system, gamepad, accelerometer, gyroscope, or other input device.

The memory 670 may contain computer program instructions (grouped as modules or components in some embodiments) that the processing unit 610 executes in order to implement one or more embodiments. The memory 670 generally includes RAM, ROM and/or other persistent, auxiliary or non-transitory computer-readable media. The memory 670 may store an operating system 672 that provides computer program instructions for use by the processing unit 610 in the general administration and operation of the computing device 600. The memory 670 may further include computer program instructions and other information for implementing aspects of the present disclosure.

For example, in one embodiment, the memory 670 includes a VNTR status determination module 674 for determining a VNTR status. The VNTR status determination module 674 can perform the methods disclosed herein. In addition, memory 670 may include or communicate with the data store 690 and/or one or more other data stores that store one or more inputs, one or more outputs, and/or one or more results (including intermediate results) of determining a VNTR status of the present disclosure, such the long reads, the plurality of haplotypes determined, the short reads, and the VNTR status (e.g., haplotypes or genotype of a sample) determined.

In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD-ROM, DVD-ROM, memory stick, flash drive, hard drive, SSD hard drive, server, mainframe storage system and the like.

In some embodiments, the methods may be written in any of various suitable programming languages, for example compiled languages such as C, C #, C++, Fortran, and Java. Other programming languages could be script languages, such as Perl, MatLab, SAS, SPSS, Python, Ruby, Pascal, Delphi, R and PHP. In some embodiments, the methods are written in C, C #, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (e.g., iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter-or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

### Additional Notes

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

1. A method for determining a score for the copy number of repeat units in a variable number tandem repeat (VNTR) locus in a target polynucleotide, comprising:
obtaining a plurality of paired-end sequence reads from a nucleic acid sequencer, wherein each paired-end sequence read is associated with a nucleic acid fragment that spans the VNTR locus in the target polynucleotide;
obtaining an alignment, against a reference sequence, of each paired-end sequence read associated with a spanning region of the VNTR locus;
determining, based on the alignment, one or more lengths of the nucleic acid fragments associated with each paired-end sequence read;
calculating a first distribution of the lengths of the nucleic acid fragments;
determining secondary distributions of the lengths of mapped segments in normalization regions for a plurality of copy numbers associated with the plurality of paired-end sequence reads; and
comparing the first distribution with at least one of the secondary distributions to generate a score of the copy number of repeat units in the VNTR locus in the target polynucleotide.

2. The method of claim 1, wherein the normalization regions are evolutionarily conserved regions in the reference sequence and/or are regions in the reference sequence that do not comprise structural variation events.

3. The method of claim 1, wherein obtaining the plurality of paired-end sequence reads comprises selecting, from a set of sequence reads, a subset of sequence reads that align to the VNTR locus with an alignment score higher than a threshold.

4. The method of claim 1, wherein the alignment tolerates a degree of mismatch lower than a threshold.

5. The method of claim 1, further comprising confirming that at least 40 paired-end sequence reads align to the VNTR locus in the reference sequence.

6. The method of claim 1, further comprising confirming that at least 50 paired-end sequence reads align to the VNTR locus in the reference sequence.

7. (canceled)

8. (canceled)

9. The method of claim 1, wherein the first distribution is compared with the at least one of the secondary distributions by way of a non-parametric probability calculation.

10. The method of claim 1, wherein the secondary distributions are determined by statistical modeling and compared to the first distribution by statistical tests.

11. The method of claim 10, wherein the secondary distributions are determined based in part on the plurality of copy numbers, the pattern of the VNTR locus, and copy number of the VNTR locus.

12. The method of claim 1, wherein comparing the first distribution with the at least one of the secondary distributions comprises comparing a posterior probability for each genotype.

13. The method of claim 1, wherein a repeat unit is longer than about 10 base pairs in length.

14. The method of claim 1, wherein the VNTR locus is about 300 base pairs to about 600 base pairs in length.

15. The method of claim 1, wherein the VNTR locus is part of a macrosatellite or a minisatellite.

16. The method of claim 15, wherein the macrosatellite has repeat patterns of longer than 100 base pairs in length.

17. The method of claim 15, wherein the minisatellite has repeat patterns of about 10 base pairs to about 100 base pairs in length.

18. The method of claim 1, wherein each paired-end sequence read is about 100 base pairs to about 500 base pairs in length.

19. The method of claim 1, wherein the plurality of paired-end sequence reads is generated by targeted sequencing, whole genome sequencing (WGS), or clinical WGS.

20. The method of claim 1, wherein the plurality of paired-end sequence reads is generated by a next generation sequencing reaction.

21-46. (canceled)

47. A system for determining a score for the copy number of repeat units in a variable number tandem repeat (VNTR) locus in a target polynucleotide, comprising:
a nucleic acid sequencer;
a non-transitory memory configured to store executable instructions; and
a hardware processor in communication with the nucleic acid sequencer and the non-transitory memory, the hardware processor programmed by the executable instructions to:
obtain a plurality of paired-end sequence reads from the nucleic acid sequencer, wherein each paired-end sequence read is associated with a nucleic acid fragment that spans the VNTR locus in the target polynucleotide;
obtain an alignment, against a reference sequence, of each paired-end sequence read associated with a spanning region of the VNTR locus;
determining, based on the alignment, one or more lengths of the nucleic acid fragments associated with each paired-end sequence read;
calculating a first distribution of the lengths of the nucleic acid fragments;
determining secondary distributions of the lengths of mapped segments in normalization regions for a plurality of copy numbers associated with the plurality of paired-end sequence reads; and
comparing the first distribution with at least one of the secondary distributions to generate a score of the copy number of repeat units in the VNTR locus in the target polynucleotide.

48. A system for determining the nucleotide sequence of a sample nucleic acid having repeat units, comprising:
a nucleic acid sequencer;
a non-transitory memory configured to store executable instructions; and
a hardware processor in communication with the nucleic acid sequencer and the non-transitory memory, the hardware processor programmed by the executable instructions to:
obtain a first plurality of paired-end sequence reads from the nucleic acid sequencer that each aligns and spans a variable number tandem repeat (VNTR) locus in a reference sequence, and a copy number of repeat units in the sample nucleic acid;
determine a consensus pattern motif and one or more positions of the first plurality of paired-end sequence reads with respect to the VNTR locus, the consensus pattern motif comprising a plurality of events of single nucleotide variants or indels;
determine, based on the consensus pattern motif and the one or more positions of the first plurality of paired-end sequence reads with respect to the VNTR locus, which repeat unit of the copy number of repeat units an event occurs in; and
construct the nucleotide sequence based in part on the copy number of repeat units, and which repeat unit of the copy number of repeat units the event occurs within.


SEQUENCE LISTING (3 sequences)

SEQ ID NO: 1 - DNA, 48 bp, Homo sapiens
  source 1..48: mol_type = genomic DNA
accccgagctagggtgcagcccggccgcactgcaggagacccaccagg

SEQ ID NO: 2 - DNA, 30 bp, Homo sapiens
  source 1..30: mol_type = other DNA
tgtatgattgacccttacgggggcctactg

SEQ ID NO: 3 - DNA, 63 bp, Homo sapiens
  source 1..63: mol_type = other DNA
tgattgagccttacggaccctttcggaccctttggacccttacggacccttaggggggcctac